(19) United States
(12) Patent Application Publication
(10) Pub. No.: US 2024/0041402 A1
(43) Pub. Date: Feb. 8, 2024

(54) Health Monitoring System

(71) Applicant: Rodrick Stevenson, Clarion, PA (US)

(72) Inventor: Rodrick Stevenson, Clarion, PA (US)

(21) Appl. No.: 17/881,309

(22) Filed: Aug. 4, 2022

(51) Int. Cl. A61B 5/00; A61B 5/282; A61B 7/04; A61B 5/0205; A61B 5/021; A61B 5/1455; A61B 5/256

(52) U.S. Cl. CPC A61B 5/6805; A61B 5/282; A61B 7/04; A61B 5/0022; A61B 5/681; A61B 5/02055; A61B 5/02141; A61B 5/14552; A61B 5/7465; A61B 5/256

(57) ABSTRACT

A health monitoring system for determining various signals based on the physiological processes of a user and transmitting them to a remote monitoring station includes a vest with a plurality of electrocardiogram (EKG) leads and a plurality of stethoscope sensors configured for determining a plurality of EKG signals and a plurality of auscultation signals respectively when the vest is worn by a user and transmitting those signals to a remote monitoring station. A wrist monitoring device is wearable on a wrist of the user and determines an oxygen saturation signal, a body temperature signal, and a blood pressure signal when the wrist monitoring device is worn on the wrist, thereafter transmitting the signals to the remote monitoring station.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to health monitoring systems and more particularly pertains to a new health monitoring system for determining various signals based on the physiological processes of a user and transmitting them to a remote monitoring station.

### (2) Description of Related Art Including Information Disclosed Under 37 CFR 1.97 and 1.98

The prior art relates to health monitoring systems which utilize a device wearable on a torso of a user for monitoring the health of the user through the use of electrocardiogram sensors and stethoscope sensors. The prior art discloses systems which monitor the health of the user in this manner and transmitting signals from the sensors to a remote location for review by a medical professional. However, the prior art does not disclose a device which utilizes the form of a vest that is concealable under most clothing and which works in tandem with a wrist device that monitors oxygen saturation, body temperature, and blood pressure of the user. The prior art also does not disclose a system which provides the above functions while also providing an alert button positioned on the wrist-worn device so that the user can alert the medical professional to a medical emergency and the professional can monitor the aforementioned health data of the user while the user experiences the medical emergency.

### BRIEF SUMMARY OF THE INVENTION

An embodiment of the disclosure meets the needs presented above by generally comprising a vest with a plurality of electrocardiogram (EKG) leads and a plurality of stethoscope sensors configured for determining a plurality of EKG signals and a plurality of auscultation signals respectively when the vest is worn by a user and transmitting those signals to a remote monitoring station. A wrist monitoring device is wearable on a wrist of the user and determines an oxygen saturation signal, a body temperature signal, and a blood pressure signal when the wrist monitoring device is worn on the wrist, thereafter transmitting the signals to the remote monitoring station.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a perspective view of a health monitoring system according to an embodiment of the disclosure.

FIG. 2 is a front view of a vest of an embodiment of the disclosure.

FIG. 3 is a rear view of a vest of an embodiment of the disclosure.

FIG. 4 is a side view of a vest of an embodiment of the disclosure.

FIG. 5 is a cross-sectional view of a vest of an embodiment of the disclosure from the arrows 5-5 in FIG. 4.

FIG. 6 is a detail cross-sectional view of a vest of an embodiment of the disclosure from circle 6 in FIG. 5.

FIG. 7 is a bottom view of a wrist monitor of an embodiment of the disclosure.

FIG. 8 is an in-use view of an embodiment of the disclosure.

### DETAILED DESCRIPTION OF THE INVENTION

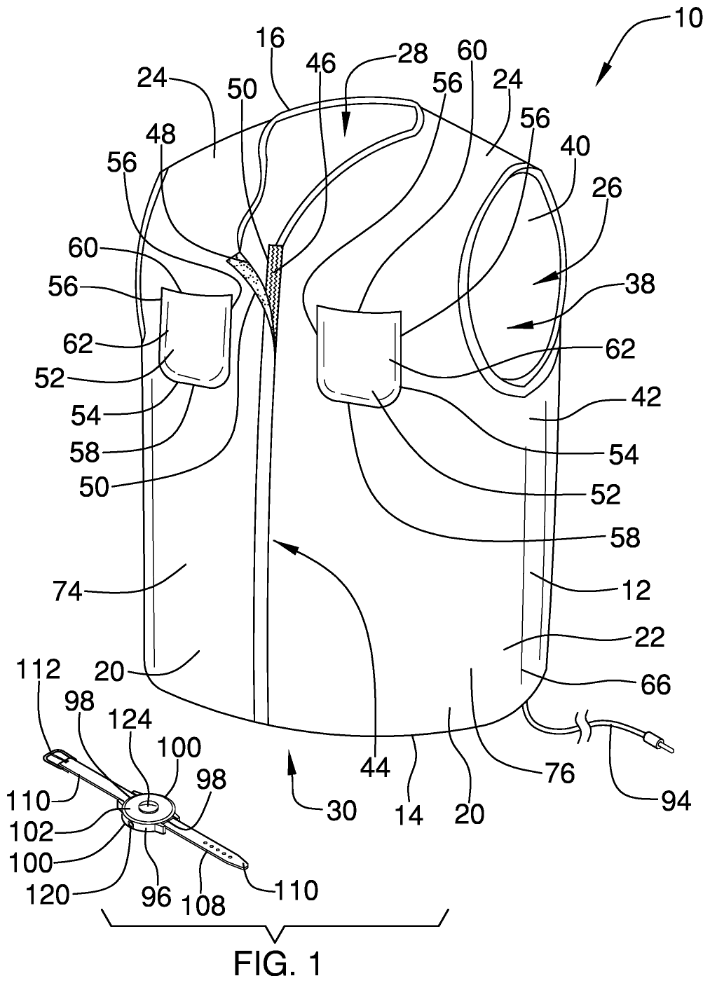

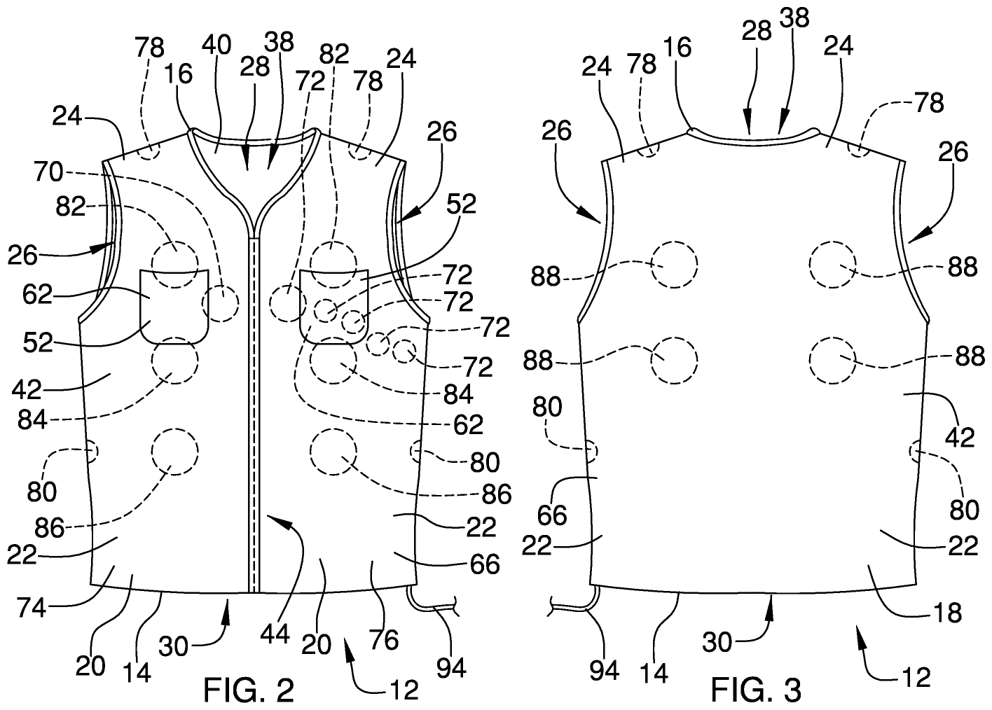

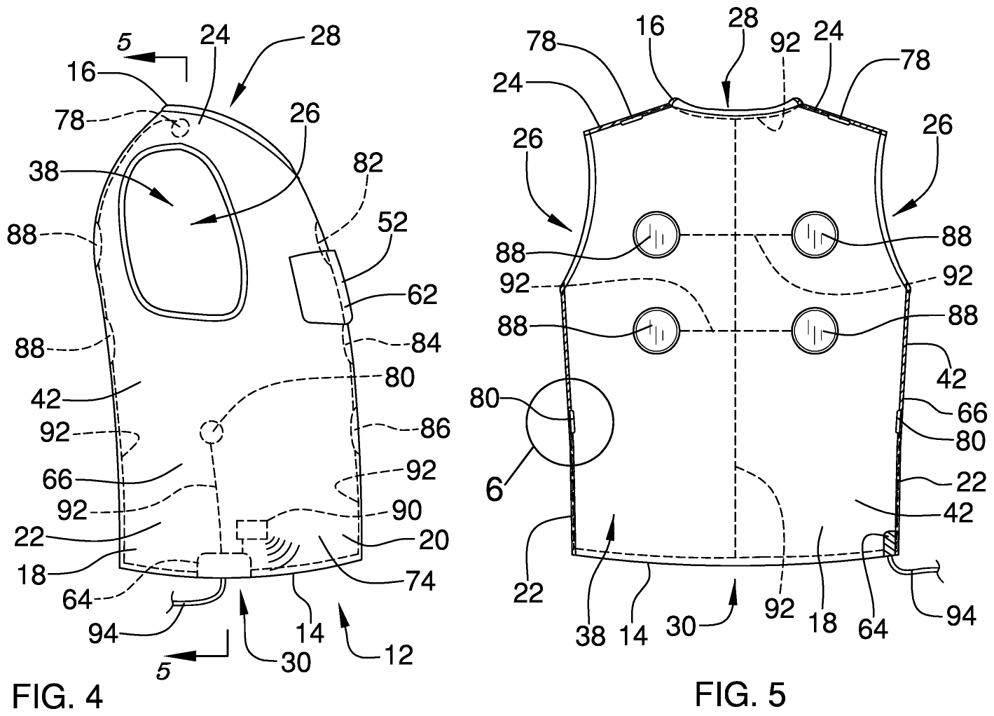

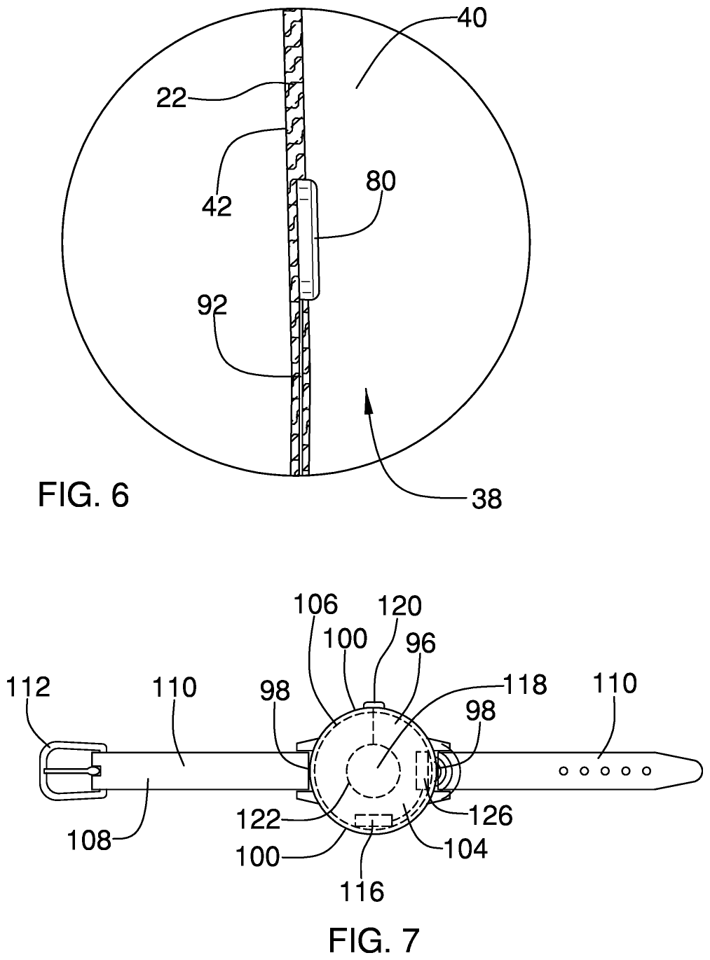

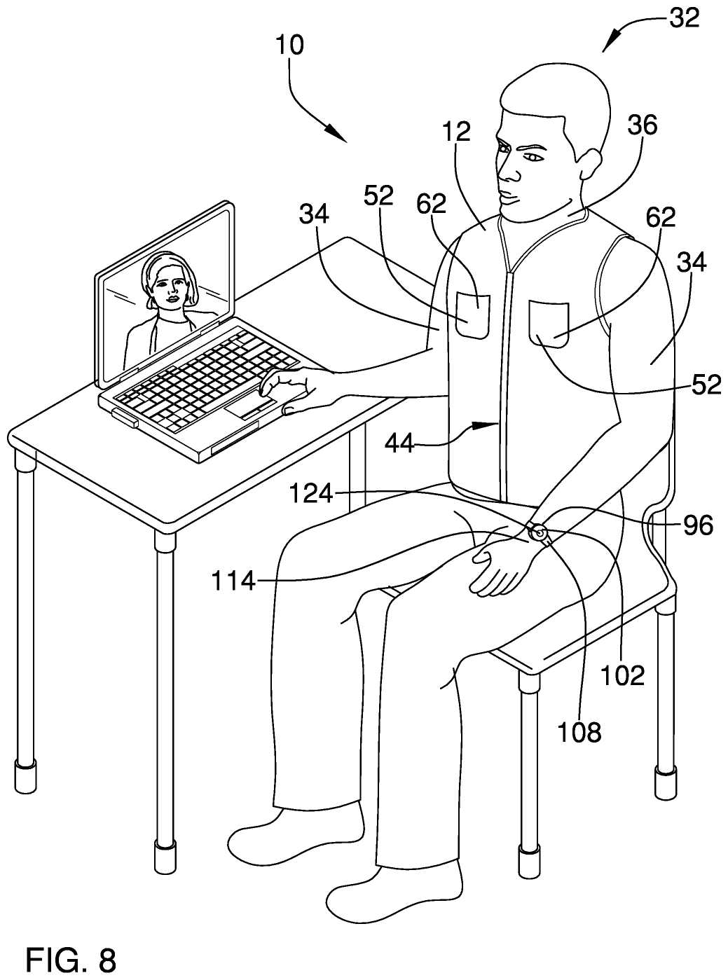

With reference now to the drawings, and in particular to FIGS. 1 through 8 thereof, a new health monitoring system embodying the principles and concepts of an embodiment of the disclosure and generally designated by the reference numeral 10 will be described.

As best illustrated in FIGS. 1 through 8, the health monitoring system 10 generally comprises a vest 12. The vest 12 has a waist end 14 and a neck end 16. The vest 12 also has a back portion 18 extending between the waist end 14 and the neck end 16. The vest 12 has a pair of front portions 20, each of which extends from the waist end 14 to the neck end 16 opposite the back portion 18, each front portion 20 being removably couplable to each other. The vest 12 has a pair of lateral side portions 22, each of which extend between a respective one of the front portions 20 and the back portion 18 adjacent the waist end 14. The vest 12 also has a pair of shoulder portions 24, each of which extends between a respective front portion 20 and the back portion 18 across the neck end 16.

Each of a pair of arm holes 26 is defined by the back portion 18, an associated shoulder portion 24 of the pair of shoulder portions 24, an associated front portion 20 of the pair of shoulder portions 24, and an associated lateral side portion 22 of the pair of lateral side portions 22. A neck hole 28 is defined by the back portion 18, the pair of shoulder portions 24, and the pair of front portions 20 when the pair of front portions 20 are coupled. A waist hole 30 is defined by the back portion 18, the pair of lateral side portions 22, and the pair of front portions 20 when the pair of front portions 20 are coupled. The vest 12 is configured for being worn by a user 32. Each arm hole 26 is configured for receiving therethrough an arm 34 of the user 32, the neck hole 28 is configured for receiving therethrough a neck 36 of the user 32, and the waist hole 30 is configured for receiving therethrough a torso of the user 32 proximate a waist of the user 32 when the vest 12 is worn. The vest 12 defines an inner space 38 and has an inner surface 40 facing the inner space 38. The vest 12 also has an outer surface 42 facing away from the inner space 38.

A hook-and-loop fastener 44 has a hook member 46 and a loop member 48. Each of the hook member 46 and the loop member 48 is coupled to an associated one of the front portions 20 of the vest 12 such that each of the hook member 46 and the loop member 48 extend along an edge 50 of the associated front portion 20 from the waist end 14 to the neck end 16. The hook member 46 is removably couplable to the loop member 48, thereby removably coupling the pair of front portions 20 to each other.

Each of a pair of pocket members 52 is coupled to the outer surface 42 of the vest 12. Each pocket member 52 is coupled to an associated front portion 20 of the vest 12 along a perimeter edge 54 of each pocket member 52 to form each of a pair of pockets 62. Each pocket member 52 is rectangular with a pair of lateral portions 56 of the perimeter edge 54, a bottom portion 58 of the perimeter edge 54 extending between the lateral portions 56 of the perimeter edge 54, and a top portion 60 of the perimeter edge 54 extending between the lateral portions 56 of the perimeter edge 54 and opposing the bottom edge 50. The pair of lateral portions 56 of each pocket member 52 and the bottom portion 58 of each pocket member 52 is coupled to the associated front portion 20. The top portion 60 of the perimeter edge 54 faces toward the neck end 16 of the vest 12. Each pocket member 52 is equidistant from the neck end 16 and is positioned closer to the neck end 16 than to the waist end 14.

A vest microprocessor 64 is coupled to the vest 12 and positioned between the inner surface 40 of the vest 12 and the outer surface 42 of the vest 12. The vest microprocessor 64 is positioned within a left lateral side portion 66 of the pair of lateral portions 56 proximate the waist end 14 of the vest 12.

Each of a plurality of electrocardiogram (EKG) leads is coupled to the inner surface 40 of the vest 12 and is configured for abutting a skin of the user 32 when the vest 12 is worn by the user 32. Each EKG lead is operatively coupled to the vest microprocessor 64 and is configured for generating and transmitting a respective EKG signal of a plurality of EKG signals to the vest microprocessor 64.

The plurality of EKG leads includes a set of six chest leads and a set of four limb leads. The set of six chest leads comprises a right chest lead 70 and a set of five left chest leads 74. The right chest lead 70 is positioned on a right front portion 74 of the pair of front portions 20 and is configured for being positioned on a chest of the user 32 proximate a sternum of the user 32 when the vest 12 is worn. The set of five left chest leads 74 is positioned on a left front portion 76 of the pair of front portions 20, wherein the left front portion 76 is coupled to the left lateral side portion 66. The set of five left chest leads 74 is configured for being positioned on the chest of the user 32. The set of four limb leads including a pair of shoulder limb leads 78 and a pair of lateral side limb leads 80. Each shoulder limb lead 78 is positioned on a respective shoulder portion 24 of the pair of shoulder portions 24 and is configured to abut an associated shoulder of a pair of shoulders of the user 32 when the vest 12 is worn. Each lateral side limb lead 80 is positioned on a respective lateral side portion 22 of the pair of lateral side portions 22 and is configured for abutting a respective side of a pair of sides of an abdomen of the user 32 when the vest 12 is worn.

Each of a plurality of stethoscope sensors is coupled to the inner surface 40 of the vest 12 and is configured for abutting the skin of the user 32 when the vest 12 is worn by the user 32. Each stethoscope sensor is operatively coupled to the vest microprocessor 64 and is configured for recording and transmitting a respective auscultation signal of a plurality of auscultation signals to the vest microprocessor 64.

The plurality of stethoscope sensors comprises a set of six front sensors and a set of four back sensors 88. The set of six front sensors including a pair of upper chest sensors 82, a pair of lower chest sensors 84, and a pair of abdominal sensors 86. One front sensor of each of the pair of upper chest sensors 82, the pair of lower chest sensors 84, and the pair of abdominal sensors 86 is positioned on the left front portion 76 of the vest 12, and another front sensor of each of the pair of upper chest sensors 82, the pair of lower chest sensors 84, and the pair of abdominal sensors 86 is positioned on the right front portion 74 of the vest 12. Each chest sensor positioned on the left front portion 76 and an associated chest sensor positioned on the right front portion 74 is equidistant to the waist end 14 of the vest 12. The chest sensors positioned on the left front portion 76 and the chest sensors positioned on the right front portion 74 are equidistant from the edge 50 of the left front portion 76 and the edge 50 of the right front portion 74 respectively.

Each upper chest sensor 82 is positioned between the plurality of EKG leads and the neck end 16 of the vest 12, and each lower chest sensor 84 is positioned opposing an associated upper chest sensor 82 of the pair of upper chest sensors 82 across the plurality of EKG leads 68. Each of the pair of upper chest sensors 82 and the pair of lower chest sensors 84 is configured for abutting the chest of the user 32 when the vest 12 is worn. Each abdominal sensor 86 is positioned between an associated lower chest sensor 84 and the waist end 14 of the vest 12 and is configured for abutting the abdomen of the user 32 when the vest 12 is worn. Each back sensor 88 is positioned on the back portion 18 of the vest 12 opposite an associated one of the pair of upper chest sensors 82 and the pair of lower chest sensors 84 across the inner space 38 and is configured for abutting a back of the user 32 when the vest 12 is worn.

A vest transmitter 90 is coupled to the vest 12 and positioned between the inner surface 40 of the vest 12 and the outer surface 42 of the vest 12. The vest transmitter 90 is positioned proximate to and operatively coupled to the vest microprocessor 64. The vest transmitter 90 is configured for receiving the plurality of EKG signals and the plurality of auscultation signals and wirelessly transmitting each of the plurality of EKG signals and the plurality of auscultation signals to a remote monitoring device. A plurality of wires 92 couples each EKG lead 68, each stethoscope sensor, and the vest transmitter 90 to the vest microprocessor 64. The plurality of wires 92 is positioned between the inner surface 40 of the vest 12 and the outer surface 42 of the vest 12. A power cord 94 is coupled to the vest microprocessor 64 and extends from the waist end 14 of the left lateral side portion 66 of the vest 12. The power cord 94 is configured for coupling to a power source, thereby activating the vest microprocessor 64.

A wrist monitor housing 96 has a pair of ends 98 and a pair of lateral surfaces 100 extending between the pair of ends 98. The wrist monitor housing 96 also has a top surface 102 and a bottom surface 104 opposing the top surface 102. Each of the top surface 102 and the bottom surface 104 extend between the pair of ends 98 and the pair of lateral surfaces 100. The wrist monitor housing 96 defines an interior space 106 within the wrist monitor housing 96. The top surface 102 and the bottom surface 104 are circular, and each lateral surface 100 is arcuate.

A wristband 108 is coupled to each end 98 of the wrist monitor housing 96 and is positionable in a loop. The wristband 108 is configured for being worn on a wrist 114 of the user 32. The wristband 108 has a pair of portions 110, each of which is coupled to an associated end 98 of the wrist monitor housing 96. Each portion 110 of the wristband 108 is removably couplable to another portion 110 of the wristband 108 via a buckle 112. A wrist monitor microprocessor 116 is coupled to and positioned within the wrist monitor housing 96.

A battery 118 is operatively coupled to the wrist monitor microprocessor 116. The battery 118 is coupled to and positioned within the wrist monitor housing 96 and is rechargeable. A charging port 120 is positioned in one of the lateral surfaces 100 of the wrist monitor housing 96. The charging port 120 is electrically coupled to the battery 118 and is configured for coupling to a power supply to charge the battery 118.

A wrist sensor 122 is positioned on the bottom surface 104 of the wrist monitor housing 96. The wrist sensor 122 is operatively coupled to the wrist monitor microprocessor 116 and is configured for abutting the wrist 114 of the user 32 when the wristband 108 is worn on the wrist 114 of the user 32. The wrist sensor 122 is configured for determining an oxygen saturation signal, a body temperature signal, and a blood pressure signal and transmitting each of the oxygen saturation signal, the body temperature signal, and the blood pressure signal to the wrist monitor microprocessor 116. An alert button 124 is positioned on and centered on the top surface 102 of the wrist monitor housing 96. The alert button 124 is operatively coupled to the wrist monitor microprocessor and is depressible, the alert button 124 transmitting an alert signal to the wrist monitor microprocessor when depressed. A wrist monitor transmitter 126 is coupled to and positioned within the wrist monitor housing 96. The wrist monitor transmitter 126 is operatively coupled to the wrist monitor microprocessor 116 and is configured for receiving each of the oxygen saturation signal, the body temperature signal, the blood pressure signal, and the alert signal from the wrist monitor microprocessor 116 and wirelessly transmitting each of the oxygen saturation signal, the body temperature signal, the blood pressure signal, and the alert signal to the remote monitoring station.

In use, the vest 12 and wrist monitor housing 96 are worn by the user 32. While wearing the vest 12 and the monitor housing, the plurality of EKG leads generates the plurality of EKG signals and the plurality of stethoscope sensors recording the plurality of auscultation signals, which are sent by the vest microprocessor 64 to the remote monitoring station. Likewise, the wrist sensor 122 determines an oxygen saturation signal, a body temperature signal, and a blood pressure signal, which are each sent by the wrist monitor transmitter 126 to the remote monitoring station. This information is then viewable by another individual, such as a trained medical professional. The user 32 can wear the vest 12 and the wrist monitor housing 96 during daily activities. If the user 32 is experiencing a medical emergency, the user 32 can depress the alert button 124, thereby wirelessly transmitting an alert signal to the remote monitoring station, at which point the other individual can respond while remotely assessing the health of the user 32.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

1. A health monitoring system comprising:
a vest, said vest being configured for being worn by a user, said vest defining an inner space, said vest having an inner surface facing said inner space, said vest having an outer surface facing away from said inner space;
a vest microprocessor, said vest microprocessor being coupled to said vest, said vest microprocessor being configured for operatively coupling to a power source;
a plurality of electrocardiogram (EKG) leads, each said EKG lead being coupled to said inner surface of said vest, each said EKG lead being configured for abutting a skin of the user when said vest is worn by the user, each said EKG lead being operatively coupled to said vest microprocessor, each said EKG lead being configured for generating and transmitting a respective EKG signal of a plurality of EKG signals to said vest microprocessor;
a plurality of stethoscope sensors, each said stethoscope sensor being coupled to said inner surface of said vest, each said stethoscope sensor being configured for abutting the skin of the user when said vest is worn by the user, each said stethoscope sensor being operatively coupled to said vest microprocessor, each said stethoscope sensor being configured for recording and transmitting a respective auscultation signal of a plurality of auscultation signals to said vest microprocessor;
a vest transmitter, said vest transmitter being coupled to said vest, said vest transmitter being operatively coupled to said vest microprocessor, said vest transmitter being configured for receiving the plurality of EKG signals and the plurality of auscultation signals and wirelessly transmitting each of the plurality of EKG signals and the plurality of auscultation signals to a remote monitoring device;
a plurality of wires, said plurality of wires coupling each said EKG lead, each said stethoscope sensor, and said vest transmitter to said vest microprocessor;
a wrist monitor housing;
a wristband, said wristband being coupled to said wrist monitor housing, said wristband being positionable in a loop, said wristband being configured for being worn on a wrist of the user;
a wrist monitor microprocessor, said wrist monitor microprocessor being coupled to and positioned within said wrist monitor housing;
a battery, said battery being operatively coupled to said wrist monitor microprocessor, said battery being coupled to said wrist monitor housing;
a wrist sensor, said wrist sensor being positioned on said wrist monitor housing, said wrist sensor being operatively coupled to said wrist monitor microprocessor, said wrist sensor being configured for abutting the wrist of the user when said wristband is worn on the wrist of the user, said wrist sensor being configured for determining an oxygen saturation signal, a body temperature signal, and a blood pressure signal and transmitting each of the oxygen saturation signal, the body temperature signal, and the blood pressure signal to said wrist monitor microprocessor;
an alert button, said alert button being positioned on said wrist monitor housing, said alert button being operatively coupled to said wrist monitor microprocessor, said alert button being depressible, said alert button transmitting an alert signal to said wrist monitor microprocessor when depressed; and
a wrist monitor transmitter, said wrist monitor transmitter being coupled to said wrist monitor housing, said wrist monitor transmitter being operatively coupled to said wrist monitor microprocessor, said wrist monitor transmitter being configured for receiving each of the oxygen saturation signal, the body temperature signal, the blood pressure signal, and the alert signal from said wrist monitor microprocessor and wirelessly transmitting each of the oxygen saturation signal, the body temperature signal, the blood pressure signal, and the alert signal to the remote monitoring station.

2. The device of claim 1, further comprising:
said vest having a waist end and a neck end, said vest having a back portion extending between said waist end and said neck end, said vest having a pair of front portions, each said front portion extending from said waist end to said neck end opposite said back portion, each said front portion being removably couplable to each other, said vest having a pair of lateral side portions, each lateral side portion extending between a respective one of said front portions and said back portion adjacent said waist end, said vest having a pair of shoulder portions, each shoulder portion extending between a respective front portion and said back portion across said neck end;
a pair of arm holes being defined by said back portion, an associated shoulder portion of said pair of shoulder portions, an associated front portion of said pair of shoulder portions, and an associated lateral side portion of said pair of lateral side portions, a neck hole being defined by said back portion, said pair of shoulder portions, and said pair of front portions when said pair of front portions are coupled, a waist hole being defined by said back portion, said pair of lateral side portions, and said pair of front portions when said pair of front portions are coupled; and
each said arm hole being configured for receiving therethrough an arm of the user when said vest is worn, said neck hole being configured for receiving therethrough a neck of the user when said vest is worn, said waist hole being configured for receiving therethrough a torso of the user proximate a waist of the user when said vest is worn.

3. The device of claim 2, further comprising:
said vest microprocessor being positioned between said inner surface of said vest and said outer surface of said vest, said vest microprocessor being positioned within a left lateral side portion of said pair of lateral portions proximate said waist end of said vest;
said vest transmitter being positioned between said inner surface of said vest and said outer surface of said vest, said vest transmitter being positioned proximate to said vest microprocessor; and
said plurality of wires being positioned between said inner surface of said vest and said outer surface of said vest.

4. The device of claim 3, further comprising a power cord, said power cord being coupled to said vest microprocessor, said power cord extending from said waist end of said left lateral side portion of said vest, said power cord being configured for coupling to a power source, thereby activating said vest microprocessor.

5. The device of claim 2, further comprising said plurality of EKG leads including a plurality of chest leads, said plurality of chest leads being positioned on an associated one of said pair of front portions, each said chest lead being configured for being positioned on a chest of the user when said vest is worn.

6. The device of claim 5, further comprising said plurality of chest leads being a set of six chest leads, said set of six chest leads comprising a right chest lead and a set of five left chest leads, said right chest lead being positioned on a right front portion of said pair of front portions, said right chest lead being configured for being positioned on a chest of the user proximate a sternum of the user when said vest is worn, said set of five left chest leads being positioned on a left front portion of said pair of front portions, said left front portion being coupled to said left lateral side portion, said set of five left chest leads being configured for being positioned on the chest of the user.

7. The device of claim 2, further comprising said plurality of EKG leads including a pair of shoulder limb leads, each said shoulder limb lead being positioned on a respective shoulder portion of said pair of shoulder portions, each said shoulder limb lead being configured to abut an associated shoulder of a pair of shoulders of the user when said vest is worn.

8. The device of claim 2, further comprising said plurality of EKG leads including a pair of lateral side limb leads, each said lateral side limb lead being positioned on a respective lateral side portion of said pair of lateral side portions, each said lateral side limb lead being configured for abutting a respective side of a pair of sides of an abdomen of the user when said vest is worn.

9. The device of claim 2, further comprising a plurality of front sensors and a plurality of back sensors, each said front sensor being positioned on an associated one of said pair of front portions, each said front sensor being configured for abutting a front portion of the torso of the user when said vest is worn, each said back sensor being positioned on said back portion of said vest, each said back sensor being configured for abutting a back of the user when said vest is worn.

10. The device of claim 9, further comprising:
said plurality of front sensors being a set of six front sensors, said plurality of back sensors being a set of four back sensors;
said set of six front sensors including a pair of upper chest sensors, a pair of lower chest sensors, and a pair of abdominal sensors, one chest sensor of each of said pair of upper chest sensors, said pair of lower chest sensors, and said pair of abdominal sensors being positioned on said left front portion of said vest, another chest sensor of each of said pair of upper chest sensors, said pair of lower chest sensors, and said pair of abdominal sensors being positioned on said right front portion of said vest;
each chest sensor positioned on said left front portion and an associated chest sensor positioned on said right front portion being equidistant to said waist end of said vest, said chest sensors positioned on said left front portion and said chest sensors positioned on said right front portion being equidistant from said edge of said left front portion and said edge of said right front portion respectively;
each of said pair of upper chest sensors and said pair of lower chest sensors being configured for abutting the chest of the user when said vest is worn, each said abdominal sensor being positioned between an associated lower chest sensor and said waist end of said vest, each said abdominal sensor being configured for abutting the abdomen of the user when said vest is worn; and
each said back sensor being positioned on said back portion of said vest opposite an associated one of said pair of upper chest sensors and said pair of lower chest sensors across said inner space.

11. The device of claim 2, further comprising a fastener, said fastener having a first member and a second member, each of said first member and said second member being coupled to an associated one of said front portions of said vest, each of said first member and said second member extending along an edge of said associated front portion from said waist end to said neck end, said first member being removably couplable to said second member, thereby removably coupling said pair of front portions to each other.

12. The device of claim 11, said fastener being a hook-and-loop fastener, said first member being a hook member, said second member being a loop member.

13. The device of claim 1, further comprising a charging port, said charging port being positioned on said wrist monitor housing, said charging port being electrically coupled to said battery, said battery being rechargeable, said charging port being configured for coupling to a power supply to charge said battery.

14. The device of claim 1, further comprising said wrist monitor housing having a pair of ends, said wrist monitor housing having a pair of lateral surfaces extending between said pair of ends, said wrist monitor housing having a top surface and a bottom surface opposing said top surface, each of said top surface and said bottom surface extending between said pair of ends and said pair of lateral surfaces, said wrist monitor housing defining an interior space within said wrist monitor housing, said top surface and said bottom surface being circular, each said lateral surface being arcuate.

15. The device of claim 1, further comprising said wristband being coupled to each end of said wrist monitor housing, said wristband having a pair of portions, each said portion of said wristband being coupled to an associated end of said wrist monitor housing, each said portion of said wristband being removably couplable to another portion of said wristband via a buckle.

16. A health monitoring system comprising:
a vest, said vest having a waist end and a neck end, said vest having a back portion extending between said waist end and said neck end, said vest having a pair of front portions, each said front portion extending from said waist end to said neck end opposite said back portion, each said front portion being removably couplable to each other, said vest having a pair of lateral side portions, each lateral side portion extending between a respective one of said front portions and said back portion adjacent said waist end, said vest having a pair of shoulder portions, each shoulder portion extending between a respective front portion and said back portion across said neck end,
a pair of arm holes being defined by said back portion, an associated shoulder portion of said pair of shoulder portions, an associated front portion of said pair of shoulder portions, and an associated lateral side portion of said pair of lateral side portions, a neck hole being defined by said back portion, said pair of shoulder portions, and said pair of front portions when said pair of front portions are coupled, a waist hole being defined by said back portion, said pair of lateral side portions, and said pair of front portions when said pair of front portions are coupled,
said vest being configured for being worn by a user, each said arm hole being configured for receiving therethrough an arm of the user when said vest is worn, said neck hole being configured for receiving therethrough a neck of the user when said vest is worn, said waist hole being configured for receiving therethrough a torso of the user proximate a waist of the user when said vest is worn,
said vest defining an inner space, said vest having an inner surface facing said inner space, said vest having an outer surface facing away from said inner space;
a hook-and-loop fastener, said hook-and-loop fastener having a hook member and a loop member, each of said hook member and said loop member being coupled to an associated one of said front portions of said vest, each of said hook member and said loop member extending along an edge of said associated front portion from said waist end to said neck end, said hook member being removably couplable to said loop member, thereby removably coupling said pair of front portions to each other;
a pair of pocket members, each said pocket member being coupled to said outer surface of said vest, each said pocket member being coupled to an associated front portion of said vest along a perimeter edge of each said pocket member to form each of a pair of pockets, each pocket member being rectangular with a pair of lateral portions of said perimeter edge, a bottom portion of said perimeter edge extending between said lateral portions of said perimeter edge, and a top portion of said perimeter edge extending between said lateral portions of said perimeter edge and opposing said bottom edge, said pair of lateral portions of each said pocket member and said bottom portion of each said pocket member being coupled to said associated front portion, said top portion of said perimeter edge facing toward said neck end of said vest, each said pocket member being equidistant from said neck end, each said pocket member being positioned closer to said neck end than to said waist end;
a vest microprocessor, said vest microprocessor being coupled to said vest and positioned between said inner surface of said vest and said outer surface of said vest, said vest microprocessor being positioned within a left lateral side portion of said pair of lateral portions proximate said waist end of said vest;
a plurality of electrocardiogram (EKG) leads, each said EKG lead being coupled to said inner surface of said vest, each said EKG lead being configured for abutting a skin of the user when said vest is worn by the user, each said EKG lead being operatively coupled to said vest microprocessor, each said EKG lead being configured for generating and transmitting a respective EKG signal of a plurality of EKG signals to said vest microprocessor, said plurality of EKG leads including a set of six chest leads and a set of four limb leads,
said set of six chest leads comprising a right chest lead and a set of five left chest leads, said right chest lead being positioned on a right front portion of said pair of front portions, said right chest lead being configured for being positioned on a chest of the user proximate a sternum of the user when said vest is worn, said set of five left chest leads being positioned on a left front portion of said pair of front portions, said left front portion being coupled to said left lateral side portion, said set of five left chest leads being configured for being positioned on the chest of the user,
said set of four limb leads including a pair of shoulder limb leads and a pair of lateral side limb leads, each said shoulder limb lead being positioned on a respective shoulder portion of said pair of shoulder portions, each said shoulder limb lead being configured to abut an associated shoulder of a pair of shoulders of the user when said vest is worn, each said lateral side limb lead being positioned on a respective lateral side portion of said pair of lateral side portions, each said lateral side limb lead being configured for abutting a respective side of a pair of sides of an abdomen of the user when said vest is worn;
a plurality of stethoscope sensors, each said stethoscope sensor being coupled to said inner surface of said vest, each said stethoscope sensor being configured for abutting the skin of the user when said vest is worn by the user, each said stethoscope sensor being operatively coupled to said vest microprocessor, each said stethoscope sensor being configured for recording and transmitting a respective auscultation signal of a plurality of auscultation signals to said vest microprocessor, said plurality of stethoscope sensors comprising a set of six front sensors and a set of four back sensors,
said set of six front sensors including a pair of upper chest sensors, a pair of lower chest sensors, and a pair of abdominal sensors, one chest sensor of each of said pair of upper chest sensors, said pair of lower chest sensors, and said pair of abdominal sensors being positioned on said left front portion of said vest, another chest sensor of each of said pair of upper chest sensors, said pair of lower chest sensors, and said pair of abdominal sensors being positioned on said right front portion of said vest,
each chest sensor positioned on said left front portion and an associated chest sensor positioned on said right front portion being equidistant to said waist end of said vest, said chest sensors positioned on said left front portion and said chest sensors positioned on said right front portion being equidistant from said edge of said left front portion and said edge of said right front portion respectively,
each said upper chest sensor being positioned between said plurality of EKG leads and said neck end of said vest, each said lower chest sensor being positioned opposing an associated upper chest sensor of said pair of upper chest sensors across said plurality of EKG leads, each of said pair of upper chest sensors and said pair of lower chest sensors being configured for abutting the chest of the user when said vest is worn, each said abdominal sensor being positioned between an associated lower chest sensor and said waist end of said vest, each said abdominal sensor being configured for abutting the abdomen of the user when said vest is worn,
each said back sensor being positioned on said back portion of said vest opposite an associated one of said pair of upper chest sensors and said pair of lower chest sensors across said inner space, said set of four back sensors being configured for abutting a back of the user when said vest is worn;
a vest transmitter, said vest transmitter being coupled to said vest and positioned between said inner surface of said vest and said outer surface of said vest, said vest transmitter being positioned proximate to and operatively coupled to said vest microprocessor, said vest transmitter being configured for receiving the plurality of EKG signals and the plurality of auscultation signals and wirelessly transmitting each of the plurality of EKG signals and the plurality of auscultation signals to a remote monitoring device;
a plurality of wires, said plurality of wires coupling each said EKG lead, each said stethoscope sensor, and said vest transmitter to said vest microprocessor, said plurality of wires being positioned between said inner surface of said vest and said outer surface of said vest;
a power cord, said power cord being coupled to said vest microprocessor, said power cord extending from said waist end of said left lateral side portion of said vest, said power cord being configured for coupling to a power source, thereby activating said vest microprocessor;
a wrist monitor housing, said wrist monitor housing having a pair of ends, said wrist monitor housing having a pair of lateral surfaces extending between said pair of ends, said wrist monitor housing having a top surface and a bottom surface opposing said top surface, each of said top surface and said bottom surface extending between said pair of ends and said pair of lateral surfaces, said wrist monitor housing defining an interior space within said wrist monitor housing, said top surface and said bottom surface being circular, each said lateral surface being arcuate;
a wristband, said wristband being coupled to each end of said wrist monitor housing, said wristband being positionable in a loop, said wristband being configured for being worn on a wrist of the user,
said wristband having a pair of portions, each said portion of said wristband being coupled to an associated end of said wrist monitor housing, each said portion of said wristband being removably couplable to another portion of said wristband via a buckle;
a wrist monitor microprocessor, said wrist monitor microprocessor being coupled to and positioned within said wrist monitor housing;
a battery, said battery being operatively coupled to said wrist monitor microprocessor, said battery being coupled to and positioned within said wrist monitor housing, said battery being rechargeable;
a charging port, said charging port being positioned in one of said lateral surfaces of said wrist monitor housing, said charging port being electrically coupled to said battery, said charging port being configured for coupling to a power supply to charge said battery;
a wrist sensor, said wrist sensor being positioned on said bottom surface of said wrist monitor housing, said wrist sensor being operatively coupled to said wrist monitor microprocessor, said wrist sensor being configured for abutting the wrist of the user when said wristband is worn on the wrist of the user, said wrist sensor being configured for determining an oxygen saturation signal, a body temperature signal, and a blood pressure signal and transmitting each of the oxygen saturation signal, the body temperature signal, and the blood pressure signal to said wrist monitor microprocessor;
an alert button, said alert button being positioned on and centered on said top surface of said wrist monitor housing, said alert button being operatively coupled to said wrist monitor microprocessor, said alert button being depressible, said alert button transmitting an alert signal to said wrist monitor microprocessor when depressed; and
a wrist monitor transmitter, said wrist monitor transmitter being coupled to and positioned within said wrist monitor housing, said wrist monitor transmitter being operatively coupled to said wrist monitor microprocessor, said wrist monitor transmitter being configured for receiving each of the oxygen saturation signal, the body temperature signal, the blood pressure signal, and the alert signal from said wrist monitor microprocessor and wirelessly transmitting each of the oxygen saturation signal, the body temperature signal, the blood pressure signal, and the alert signal to the remote monitoring station.
